(19) United States
(12) Patent Application Publication
(10) Pub. No.: US 2002/0090612 A1
(43) Pub. Date: Jul. 11, 2002

(54) METHOD OF IDENTIFYING NUCLEIC ACIDS

(76) Inventors: JONATHAN M. ROTHBERG, GUILFORD, CT (US); MICHAEL MCKENNA, NEW HAVEN, CT (US); PAUL PREDKI, ANTROCH, CA (US); ANDREAS WINDMUTH, WOODBRIDGE, CT (US); RICHARD A. SHIMKETS, WEST HAVEN, CT (US)

Correspondence Address: MINTZ LEVIN COHN FERRIS GLOVSKY POPECO, ONE FINANCIAL CENTER, BOSTON, MA, 02111

(21) Appl. No.: 09/417,386

(22) Filed: Oct. 13, 1999

Related U.S. Application Data
(60) Provisional application No. 60/115,109, filed on Jan. 8, 1999.

(51) Int. Cl. C12Q 1/68; C07H 21/02; C07H 21/04

(52) U.S. Cl. 435/6; 536/23.1

(57) ABSTRACT

Disclosed are methods for identifying nucleic acids in a sample of nucleic acids in which nucleic acids are initially present in unequal amounts. The methods include partitioning the starting population of nucleic acids to form one or more subpopulations, and then identifying nucleic acids that are present in different amounts in the partitioned nucleic acid sample as compared to the starting population.

### RELATED APPLICATIONS

This application claims priority to U.S. Ser. No. 60/115,109, filed Jan. 8, 1999, which is incorporated herein in its entirety.

### FIELD OF THE INVENTION

The present invention relates to nucleic acids and more particularly to methods of equalizing the representation of nucleic acids in a population of nucleic acid molecules.

### BACKGROUND OF THE INVENTION

Approximately 10,000-20,000 genes are thought to be expressed within living cells, depending upon the specific cell type. RNAs corresponding to different genes can be present in different levels in cells. For example, transcripts from as few as 10-15 genes may represent 10-15% of cellular mRNA by mass. In addition to these highly abundant transcripts, another 1000-2000 genes encode moderately abundant transcripts, which can account for up to 50% of cellular mRNA mass. Transcripts from the remaining genes fall into the low abundance class.

Because many genes are identified by isolating complementary DNA (cDNA) corresponding to an RNA sequence, a significant problem can arise because of differences in the levels at which specific RNAs are present in cell types. The most abundant sequences can be repeatedly sampled, while the lowest abundance class may be rarely, if ever, sampled.

Several normalization and subtractive hybridization protocols have been developed to help overcome this problem. These techniques can be technically difficult to perform, and they can fail to detect cDNAs corresponding to rare transcripts.

### SUMMARY OF THE INVENTION

The invention is based in part on the discovery of novel procedures for equalizing, or normalizing, the representation of nucleic acids in a sample of nucleic acids in which different nucleic acids are initially present in the sample in unequal amounts.

Accordingly, in one aspect the invention provides a method of screening a population of nucleic acid sequences. The method includes providing a population of nucleic acid sequences, partitioning the population into one or more subpopulations of nucleic acids, and identifying a first nucleic acid sequence having an increased level in the subpopulation relative to its level in the starting population of nucleic acids. The first nucleic acid is then compared to a reference nucleic acid sequence or sequences. The absence of the first nucleic acid sequence in the reference nucleic acid or nucleic acid sequences indicates the first nucleic acid is a novel nucleic acid sequence.

The RNA can be derived from a plant, a single-celled animal, a multi-cellular animal, a bacterium, a virus, a fungus, or a yeast. If desired, the RNA can also be partitioned prior to synthesizing cDNA.

Among the advantages of the methods are that they eliminate, or minimize, redundant identification and characterization of identical nucleic acid sequences in a population of nucleic acids.

In some embodiments, the cDNA is synthesized to selectively generate cDNA species that are enriched for those sequences oriented towards the 5′-terminus of the cDNA. In other embodiments, the cDNA is synthesized to enrich for those sequences oriented towards the 3′-terminus of the cDNA.

In some embodiments, the population is normalized by digesting the cDNAs with one or more restriction endonucleases, in different reaction vessels, so as to generate segregated multiple partitions. Preferably, each specific digested cDNA-fragment will occur in only one partition.

In some embodiments, the cDNAs are partitioned by physical methods, which may optionally follow the restriction endonuclease digestion. The physical methods separate the cDNAs a function of their terminal nucleotide sequences, overall length and migratory pattern on a sizing matrix that possesses the ability to separate molecules as a function of their physical and/or biochemical properties.

In other embodiments, the cDNAs are partitioned during subsequent PCR-based amplification of adapter-ligated cDNA fragments that have been digested with one or more restriction endonucleases.

In other embodiments, the cDNAs are partitioned by screening the original mixture of cDNAs so as to remove those sequences that have already been characterized. Screening occurs using partitioned subtraction, whereby the original cDNAs are brought into contact with a prepared, subtraction library of known sequence in such a way that any sequence contained within the original library that is complimentary to any element of the subtraction library is removed or suppressed.

cDNA sequences may also be partitioned by determining the size of each cDNA fragment prior to sequencing; biasing for formation of larger fragment PCR products by lariat formation. In this method, a bias for the larger fragment within the PCR reaction is introduced to allow efficient preferential amplification of longer fragments. Alternatively, partitioning may occur by preferentially amplifying 5′ terminal or 3′ terminal sequences of mRNA molecules.

If desired, the amplified cDNAs may fractioned by separating the amplified cDNAs on a sizing matrix that separates molecules as a function of their physical and/or biochemical properties and excising individual cDNA fragments from said sizing matrix. The excised cDNA fragments are then inserted into a recombinant vector, or further amplified.

In some embodiments, the restriction endonuclease is a restriction endonuclease that possesses a recognition sequence 4 to 8 basepairs in length and produces either a 5′- or 3-terminal overhang 0 to 6 basepairs in length.

In some embodiments, the identified sequence is subjected to computational analysis. The computational analysis can include querying, or searching, a nucleotide sequence database to identify sequences that match, or the absence of any sequences that match. The database includes a plurality of known nucleotide sequences of nucleic acids that may be present in the sample.

Preferably, the nucleic acid database comprises substantially all the known, expressed nucleic acid sequences derived from a group comprising a plant, a single-celled animal, a multi-cellular animal, a bacterium, a virus, a fungus, or a yeast.

In some embodiments, sizing includes diluting and re-amplification of the cDNAs, fractionating the re-amplified cDNAs by use of one or more sizing matrixes that separate the molecules as a function of their physical and/or biochemical characteristics, physically dividing or cutting the sizing matrixes into a plurality of sections, wherein each section is comprised of one or more cDNAs of similar molecular weight or size. The cDNAs are eluted from each of the sizing matrix section, ligated into a cloning vector and transformed into a host, e.g., a bacterial host. A plurality of the transformed host colonies are selected so as to ensure a statistically-accurate representation of the cDNAs originally contained within the sizing matrix sections. The inserts from this plurality of colonies are recovered and their molecular weight or size of are determined. A plurality of insert DNAs, wherein each successive insert has a molecular weight or size that is within a 0.2 basepair window; and wherein only those DNA species that fall within the 0.2 basepair window is subsequently subjected to nucleotide sequencing.

As utilized herein, the term “normalized” is defined as a mixture of mRNAs (or cDNAs thereof) in which the copy number of highly abundant mRNA species is reduced relative to its copy number in a starting population of nucleic acids, and the copy number of a less abundant mRNA species has been enriched relative to the copy number of the latter mRNA in the starting population.

Among the advantages provided by the present invention are that it multiple partitioning strategies function in a synergistic manner so as to ameliorate unnecessary, redundant sequencing of the same sequence(s), while concomitantly enhancing the sequencing of rarer sequences.

The partition strategies disclosed herein also normalize cDNA abundance by separating the cDNA sequences into multiple partitions possessing minimal sequence overlap. In addition, the various partitioning strategies are performed so as to assure that substantially all cDNAs are sampled. An additional normalization effect may be obtained by separating the resulting DNA fragments based upon their overall size (i. e., size fractionation). Moreover, it is also possible to normalize the abundance of the cDNAs to an even greater degree by the use of one of several disclosed pre-characterization methods.

All technical and scientific terms used herein have the same meanings commonly understood by one of ordinary skill in the art to which this invention belongs. Although any methods and materials similar or equivalent to those described herein can be used in the practice of the present invention, the preferred methods and materials are now described. The citation or identification of any reference within this application shall not be construed as an admission that such reference is available as prior art to the present invention. All publications mentioned herein are incorporated herein in their entirety by reference.

### BRIEF DESCRIPTION OF THE DRAWINGS

FIG. 1 is a flow diagram illustrating a method for normalizing the abundance of nucleic acid molecules in a population of nucleic acid molecules.

FIG. 2 is a flow diagram illustrating a method of 5′-enriched cDNA synthesis according to the invention.

FIG. 3A is a schematic diagram showing restriction enzyme digestion and adapter ligation for enrichment of 5′ ends of mRNA molecules.

FIG. 3B is a histogram showing the regions of genes covered by clones constructed using 5′ end enrichment.

FIG. 3C is a schematic diagram showing restriction enzyme digestion and adapter ligation for enrichment of mRNA molecules containing internal restriction fragments.

FIG. 3D is a histogram showing the regions of genes covered by clones constructed using enrichment for internal restriction fragments.

FIGS. 4A and 4B are schematic illustrations showing the effects of partitioning on the types of nucleic acids recovered in relation to the abundance of the mRNA molecules.

### DETAILED DESCRIPTION OF THE INVENTION

The present invention provides methods for identifying nucleic acids in a population of nucleic acid samples. It is based in part on normalizing the representation of sequences that may be initially present in different levels in the population of nucleic acid sequences. The normalization takes place by one or more methods of partitioning the nucleic acid population.

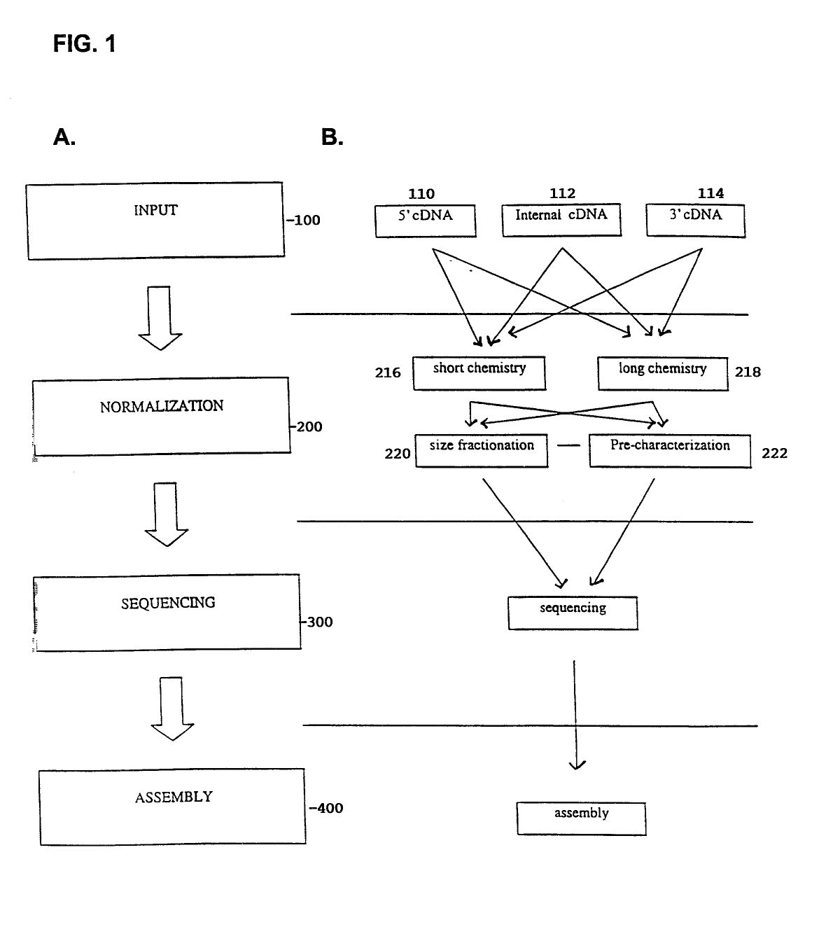

A schematized overview of the invention is shown in FIG. 1. At the input step 100 a starting population of RNA is chosen for analysis. Unless indicated otherwise, reference to a given RNA or population of RNAs is understood to also encompass reference to the corresponding cDNA or cDNAs.

Any population of RNA molecules can be used as long as the population contains, or is suspected of containing, two or more distinct RNA molecules. The population can be isolated from a starting sample using standard methods for isolating RNA. The RNA population can be isolated from, e.g., an entire organism or multiple organisms, or from a tissue or cell of an organisms. The RNA can also be isolated from, e.g., cultured cells, such as eukaryotic or prokaryotic cells grown in vitro. If desired, the RNA can be mRNA, (e.g., polyA+RNA), or stable RNAs (e.g., ribosomal RNA, transfer RNA, or small nuclear RNA). The input RNA or cDNA can be a subpopulation containing the 5′ end of RNA molecules (110), a subpopulation having an internal regions of starting RNA molecules (112), or subpopulations containing the 3′ end of the cDNA molecules (114).

The selected population or subpopulation is next subjected to a normalization analysis (200). The normalization analysis includes one or more partitioning steps that decrease the relative amount of sequences that are abundant in the starting population of nucleic acids and increase the relative representation of sequences that are rare in the starting population of nucleic acids. A partitioning step can take place before or after mRNA is converted to cDNA. A partitioning step can also take place following amplification of a cDNA. Unless stated otherwise, any partitioning method described herein can be used in conjunction with one or more additional partitioning methods. Examples of suitable partitioning steps are provided below.

In some embodiments, cDNA molecules are subjected to digestion with restriction enzymes, after which adapter oligonucleotides are ligated to the digestion products, and the resulting products amplified. FIG. 1 indicates two types of digestions and adapter ligations which can be performed. The first, designated short chemistry (216) because it tends to result in shorter amplification products, uses two restriction enzymes, followed by ligation of adapter oligonucleotides having termini complementary to the termini of the internal digestion fragments. The second, designated long chemistry (218), similarly uses restriction digestion and adapter ligation but uses longer adapters, which generally result in longer amplification products.

FIG. 1 also illustrates that the modified cDNAs can be subjected to size fractionation (220), which is an example of a partitioning method, and that information from the size fraction analysis can be used in a precharacterization analysis (222). A precharacterization can include, e.g., comparing the size of the insert to sequence databases of fragments sizes produced by the restriction enzyme. Amplification of short and long chemistry fragments can also be performed in association with partitioning steps, which are explained in detail below.

The amplified products are next sequenced (300). Sequencing can be performed by any method known in the art. The compiled sequence data are then assembled (400), and the sequence generated is compared to known sequences, e.g., sequences in publicly available databases.

The methods herein described are therefore useful for identifying genes, e.g., expressed genes in an organism of interest, e.g., a human. The sequence information obtained is particularly useful for identifying genes transcribed at low levels, or generating low levels of steady state transcripts. The methods can also be used, e.g., to identify secreted proteins for potential therapeutic use and/or for drug targets; identify variations within the human genome, such as single nucleotide polymorphisms (SNPs); identify differences between normal and diseased tissue; and analyze differential gene expression in different tissues and/or species.

Partitioning Prior to cDNA Synthesis

One approach to normalize levels of MRNA from a given sample, e.g. a given cell or tissue type, is to arbitrarily separate a starting population of RNA molecules into many smaller subpopulations, or collections. In general, a greater number of partitions increases the likelihood that a given partitions will lack a sequence or sequences that is abundant in the starting population of nucleic acid sequences. This method therefore allows for access to sequences that are expressed in very low copy number.

Alternatively, RNA populations can be isolated from different cell types. This partitioning strategy is based on the premise that different tissues tend to express different subsets of genes. Thus, RNA sequences can be partitioned by sequencing multiple different cDNA libraries extracted from one or more tissues within the body. However, the partitioning will not typically be complete, because many genes are expressed in more than one tissue type.

Synthesis and Amplification of cDNA Molecules

Typically, partitioning is performed on cDNA populations that have been modified for subsequent analysis. The modifications may include: (i) digesting the cDNA with at least one restriction endonuclease; (ii) ligating an adapter oligonucleotide to one or more ends of the termini of the digestion products; and (iii) amplifying the ligated products, e.g., in PCR-mediated amplification. These methods are particularly suited to cDNA molecule that have been constructed from the 5′, internal, and 3′ subpopulation of RNA molecules as described above. These manipulations are collectively known as SeqCalling™ chemistry. In preferred embodiments, cDNA is generated from populations of RNA molecules that have been divided into subpopulations containing 5′ ends of transcripts, populations of molecules containing internal regions of RNA molecules, or subpopulations containing 3′ ends of RNA molecules.

A. Construction and amplification of cDNA subpopulation enriched for the 5′ ends of mRNA molecules

5′-enriched cDNA synthesis generates cDNA species that are enriched for those sequences oriented towards the 5′-terminus of the cDNA, and in which a specific oligonucleotide sequence is ligated to the 5′-terminus. Approaches for generating cDNAs specifically enriched in transcript 5′ ends are often based on the synthesis of a homopolymeric (e.g., dG or dA) tail by the enzyme terminal deoxynucleotidyl transferase (TdT) subsequent to the synthesis of the first cDNA strand. Second strand synthesis is then primed by the use of a complementary homo-oligonucleotide primer sequence. See e.g., Frohman, et al., 1988. Proc. Natl. Acad. Sci. USA 85: 8998-9002; Delort, et al., 1989. Nucl. Acids Res. 17: 6439-6448; Loh, et al., 1989. Science 243: 217-220; Belyavsky, et al., 1989. Nucl. Acids Res. 17: 2919-2932; Ohara, et al., 1989. Proc. Natl. Acad. Sci USA 86: 5673-5677.

Alternatively, amplification can exploit the 5′-terminal cap structure present in eukaryotic mRNAs (see e.g., Furuichi & Miura, 1975. Nature 253: 374-375; Banerjee, 1980. Microbiol. Rev. 44: 175-205; Shatkin, 1985. Cell 40: 223-224). However, MRNA preparations generally include a mixture of both capped and non-capped mRNA species. The non-capped mRNAs are thought to be primarily the result of degradation within the cell or during the isolation procedure. An alternative approach to enrich for full-length mRNAs is to purify capped mRNA using affinity reagents. These reagents include naturally occurring proteins that bind the cap structure (see e.g., Edery, et al., 1995. Mol. Cell. Biol. 15: 3363-3371); anti-cap antibodies (see e.g., Bochnig, et al., 1987. Eur J Biochem. 68: 460-467); and chemical modification of the cap, followed by selection for the modified cap structure (see e.g., Carninci, et al., 1996. Genomics 37: 327-336). In addition, 5′-oligo capping can also be used, in which specific oligonucleotide sequences are selectively added to 5′-capped mRNAs prior to first strand cDNA synthesis. Subsequent synthesis of the second strand, is primed by an oligonucleotide that is complementary to the modified cap sequence. See e.g., Maruyama & Sugano, 1994. Gene 138: 171-174; Suzyki, et al., 1997. Gene 200: 149-156; Fromont-Racine, et al., 1993. Nucl. Acids Res. 21: 1683-1684; U.S. Pat. No. 5,597,713).

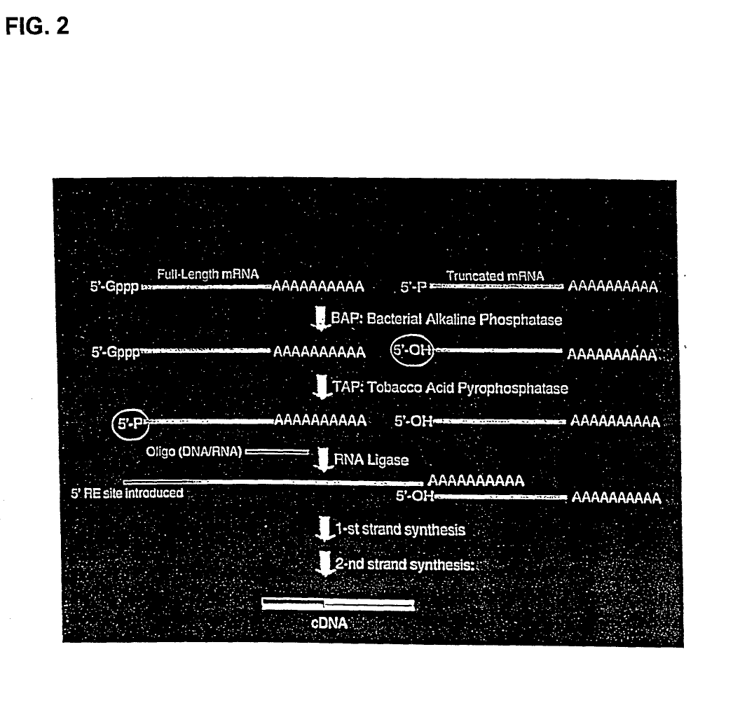

An alternative method for isolating RNA molecules containing a capped 5′ end is shown in FIG. 2. FIG. 2 depicts a flow diagram for 5′-enriched cDNA synthesis using a full-length mRNA having a 5′-terminal cap sequence (Gppp) and a poly A+tail. Also shown in FIG. 2 is truncated mRNA having a 5′ terminal phosphate group. Typically, RNA preparations contain a mixture of full-length capped RNAs and truncated mRNAs. The truncated RNAs can arise, e.g., by intracellular degradation of the RNA or by degradation of the RNA during its isolation.

In the first step in FIG. 2, the free 5′-terminal phosphate groups of the truncated or degraded mRNAs are removed by the action of a phosphatase, e.g., the bacterial alkaline phosphatase shown, or calf intestinal alkaline phosphatase. The phosphatase is then inactivated. In the second step, the 5′ cap is removed from the full-length mRNA using a pyrophosphatase, e.g., the tobacco acid pyrophosphatase shown in FIG. 2. The resulting product is the decapped full-length RNA with a free 5′-terminal phosphate group.

In the third step in FIG. 2 , the phosphate group serves as a substrate for an RNA ligase-mediated reaction that attaches a specific DNA/RNA hybrid to the 5′-terminus of the full-length mRNAs. An RNA containing the ligated hybrid is used as a substrate for first and second strand cDNA synthesis. Preferably, a combination of oligo(dT)- and random hexamer-mediated first strand priming is performed in the presence of E. coli ligase to enhance overall cDNA length. Preferably, an RNase and thermal cycling are used to remove the RNA strand after first strand synthesis. The resulting single strand DNA (ssDNA) functions as a more effective reagent for the priming of second strand synthesis.

Although first strand synthesis occurs for both types of mRNA species (i.e., full-length and truncated/degraded), only those mRNAs with the appropriate sequence ligated to the 5′-terminus (i.e., full-length mRNAs) contain a priming site for subsequent second strand synthesis. Thus, RNAs derived from the full-length mRNAs are selectively amplified.

Preferably, a thermostable enzyme for second strand synthesis in a non-thermal cycled temperature profile is used to ensure more stringent priming of the second strand reaction compared to a non-thermostable enzyme.

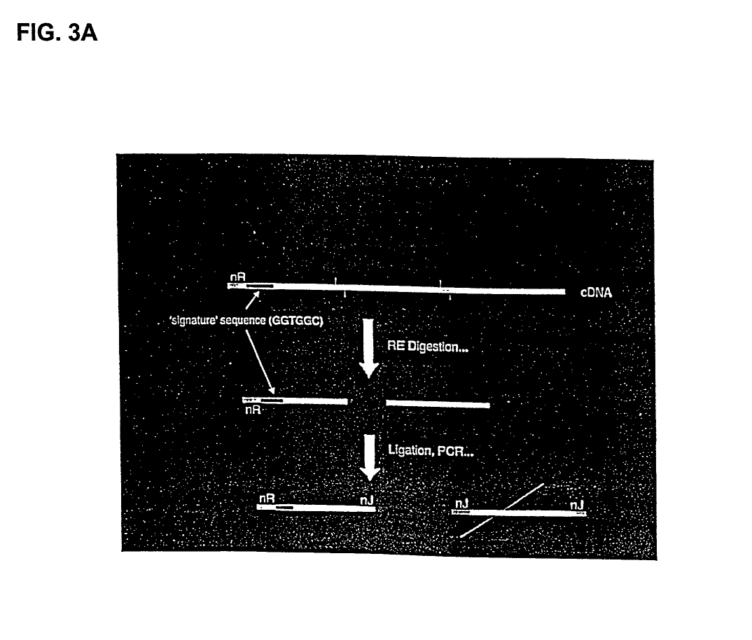

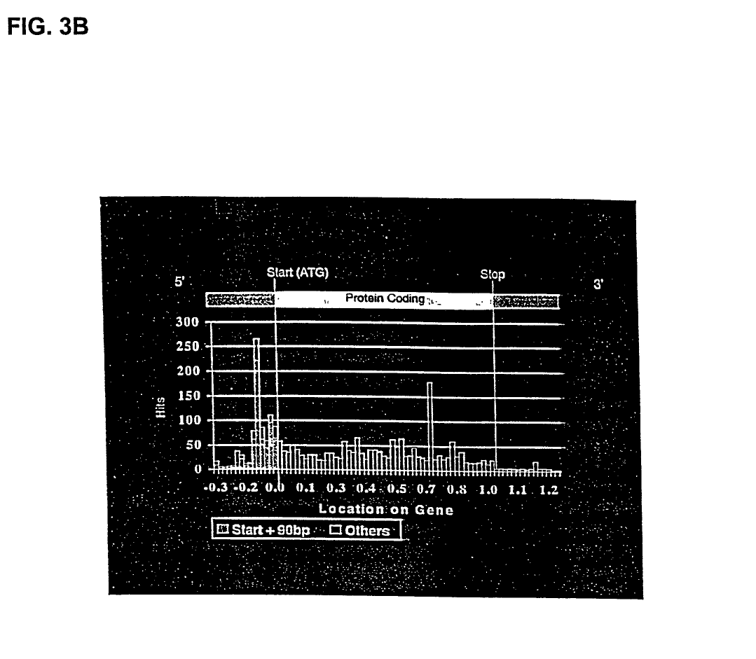

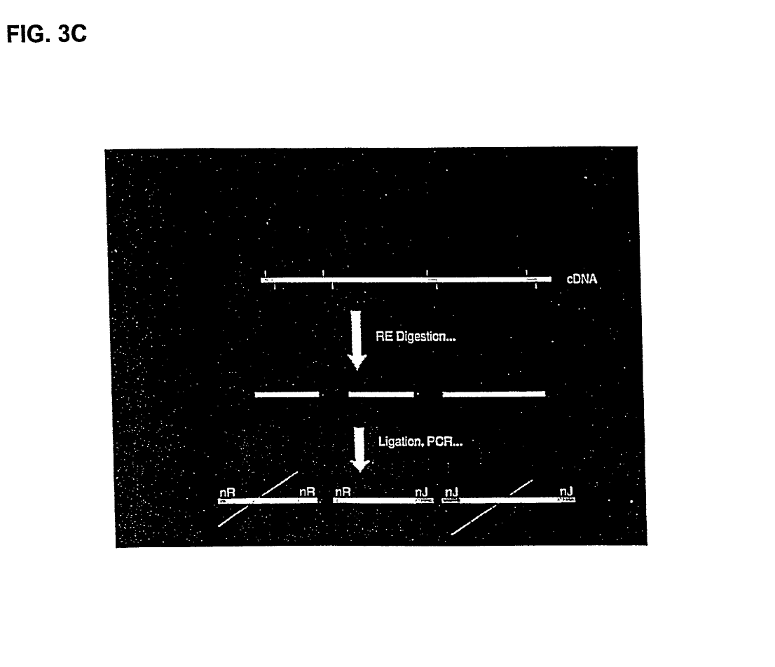

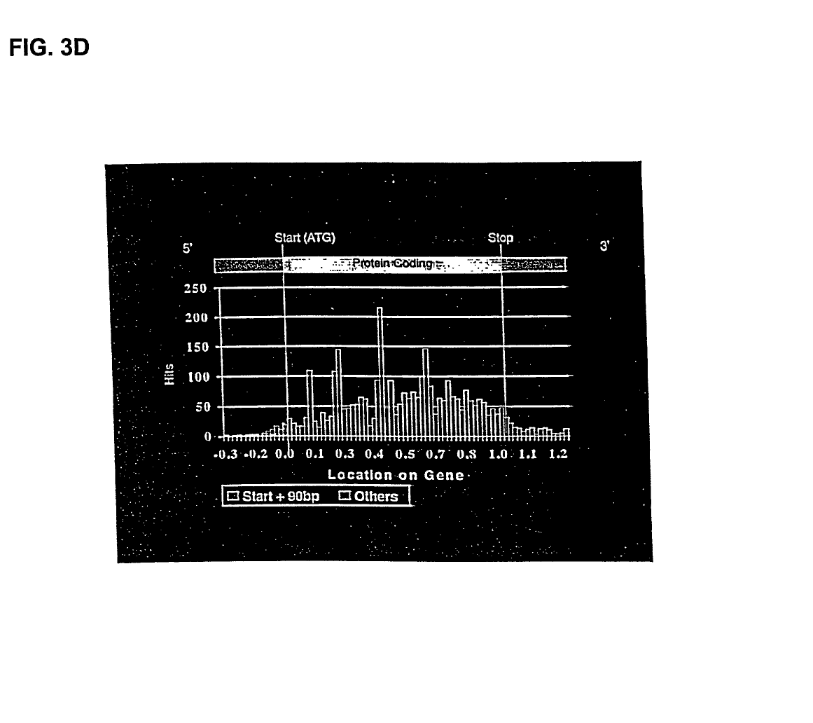

A double-stranded cDNA prepared with an adapter containing an oligonucleotide sequence (nR plus “signature sequence”) ligated to the 5′-terminus is digested with a restriction endonuclease as shown in FIG. 3A. The oligonucleotide RS [SEQ ID NO:1] (or nR) is used to prime the PCR amplification step subsequent to the ligation of the restriction digestion products. The nJ/nJ PCR product is shown as lined-through to denote that it does not clone efficiently in E. coli.

A representation of the distribution of clones derived using 5′ enriched synthesis with respect to the region of the gene they include is shown in FIG. 3B. A reference mRNA containing a 5′ terminus, an ATG initiation codon, a Stop codon, and a 3′ terminus is shown along the X-axis. Also shown is a histogram showing the number of clones (Y-axis) containing sequences derived from the indicated regions of the reference mRNA. The histogram reveals that the 5′ enrichment method method generates distributions enriched in 5′ end fragments, and has increased proportions of fragments containing the start codon and the adjacent 90 bp of coding sequences.

B. Construction and amplification of cDNA subpopulations enriched for the interior regions ends of RNA molecules

To generate relatively short cDNA fragments generated from the interior regions of a RNA molecule, i.e., from a region not containing the 5′ or 3′ terminus, the following procedure is used.

RNA is purified using any standard procedure (see e.g., Berger, 1987. Methods Enzymol 152: 215-219) and cDNA is synthesized according to standard protocols, such as random oligomer or oligo-dT primed synthesis (see, e.g., Gubler & Hoffman, 1983, Gene 25: 263-269, Okayama & Berg, 1982, Mol. Cell Biol. 2: 161-170).

The cDNA is initially digested with a pair of restriction endonucleases. Although any enzyme pair that generates distinct 5′-terminus overhangs is acceptable, a preferred embodiment utilizes enzymes that possess a 4-8 basepair (bp) recognition site yielding a 0-6 bp 5′-terminal overhang, and a more preferred embodiment utilizes enzymes that possess a 6 bp recognition sequence and generates a 4 bp 5′-terminus overhang. One form of manipulation for generating internal fragments is shown in FIG. 3C. The cDNAs are digested with two restriction endonucleases, yielding three types of fragments (two “homo”, one “hetero” termini). Following digestion, specific adapters are ligated and the fragments are PCR amplified based upon the specific adapter sequence utilized. As indicated by the crossed lines, the nR-nR and nJ-nJ fragments are unstable in E. coli, and are rarely observed following cloning.

Two suitable 24 nucleotide adapter molecules can be generated from RA24 [SEQ ID NO:9]; RC24 [SEQ ID NO:10]; JA24 [SEQ ID NO:11]; or JC24 [SEQ ID NO:12]. The adapters are generated by annealing the RA24, RC24, JA24 or JC24 24-mer oligonucleotides [SEQ ID NOs:9-12, respectively] with 12-mer oligonucleotides possessing sequences that are complementary to the last 8 nt of the 3′-terminus of the 24-mer and the 4 bp overhang. The sequences of these primers and other primers described herein are provided in Table 1.

These 4 bp overhang sequences are chosen so as to be complementary to the overhangs that are generated by the restriction endonuclease digestions. In addition, the last 3′-terminal nucleotide of the 24-mer adapter (i. e., A or C) is selected such that a functional restriction endonuclease recognition site is not re-generated when the adapter anneals to the digested cDNA.

Following ligation of the adapters, the restriction endonucleases are heat-inactivated, and the reaction mixture is PCR amplified.

Internal fragments may alternatively be generated using a second type of adapters, which results in longer amplified fragments (also referred to as “Long Internal Chemistry” or “Long Chemistry”). This method is similar to short chemistry, except all adapters possess an additional common sequence on their 5′-termini. This technique suppresses the amplification of small fragments while concomitantly increasing the amplification of longer fragments. The subsequent PCR amplification with the “X” and “J” primers results in production of both a hetero (i. e., “RX—JR”) adapter fragment and “homo” adapter fragments (i.e., “RX—XR” and “RJ—JR”), which are unstable in a host and are rarely observed following the cloning process.

The effectiveness of enriching for internal fragments is shown in FIG. 3D. Several thousand sequences generated from internal cDNA fragments and compared against a database of approximately 5000 known genes with annotated start and stop sites. Each sequence matching the database was assigned a location on the gene relative to the start (0.0) and stop (1.0) locations relative to the location of the 5′-most matching nucleotide (of the gene). The distribution from a standard run shows that most fragments are located “internally” (i.e., within the coding region). Fragments covering the start codon plus an additional 90 bp (located immediately 3′ of the start codon) are significant, because they have a high probability of containing enough sequence to identify secreted proteins. A small but significant fraction of the fragments covers the start codon and the additional 90 bp.

Following digestion, adapters are ligated to these 5′-terminal overhangs. The primers are longer relative to primers used to generate short fragments. Two specific pairs of adapter molecules that can be used in long chemistry synthesis include RXC [SEQ ID NO:2]; RXA [SEQ ID NO:3]; RJC [SEQ ID NO:4]; or RJA [SEQ ID NO:5]. The adapters are generated by annealing RXC, RXA, RJC or RJA oligonucleotides [SEQ ID NOs:2-5, respectively] with 12-mer oligonucleotides possessing sequences that are complementary to the last 8 nt of the 3′-terminus of the 24-mer and the 4 bp overhang. These 4 bp overhang sequences are chosen so as to be complementary to the overhangs that are generated by the restriction endonuclease digestions. In addition, the last 3′-terminal nucleotide of the 24-mer adapter (i.e., A or C) is selected such that a functional restriction endonuclease recognition site is not re-generated when the adapter anneals to the digested cDNA.

Following the ligation of the adapters, the restriction endonucleases are heat inactivated and the reaction mixture is PCR amplified. While the sequences of the two adapters are distinct, they nevertheless possess common 5′ sequences that allow the formation of lariat or pan-handle structures that function to suppress PCR-mediated amplification of the shorter fragments.

C. cDNA Synthesis of molecules enriched for 3 ′ ends

3′-enriched cDNA synthesis generates cDNAs that are enriched for the sequences oriented towards the 3′-terminus of the cDNA. This is accomplished by synthesis of the first-strand using a specific oligonucleotide sequence that has been modified to contain an adapter sequence at its 5-terminus [SEQ ID NO:14]. Following first-stand cDNA synthesis with the primer, standard cDNA synthesis protocols are utilized as illustrated in FIG. 2.

The 3′-enriched cDNA is digested with one restriction endonuclease. Although any enzyme that generates a distinct 5′-terminus overhang is acceptable, it is generally most preferred to utilize an enzyme that possesses a 6 bp recognition site yielding a 4 bp 5′-terminal overhang. Following digestion, an adapter is then ligated to these 5′-terminal overhangs. These adapters are generated from the JA24 [SEQ ID NO:11] or JC24 [SEQ ID NO:12] 24-mer annealed with 12-mer oligonucleotides possessing sequences that are complementary to the last 8 nt of the 3′-terminus of the 24-mer and the 4 bp overhang. These 4 bp overhang sequences are chosen so as to be complementary to the overhangs that are generated by the restriction endonuclease digestions. In addition, the last 3′-terminal nucleotide of the 24-mer adapter (i.e., A or C) is selected such that a functional restriction endonuclease recognition site is not re-generated when the adapter anneals to the digested cDNA.

Following the ligation of the adapters, the restriction endonucleases are heat inactivated and the reaction mixture is PCR amplified.

Longer fragments enriched for the 3′-ends can be obtained by ligating a longer primer to cDNA molecules that have been digested with a restriction enzyme. Any enzyme that generates a distinct 5′-terminus overhang can be used. It is generally preferred to utilize an enzyme that possesses a 6 bp recognition site yielding a 4 bp 5′-terminal overhang. Following digestion, an adapter is then ligated to the 5′-terminal overhangs. Acceptable adapters are generated from the JA24 [SEQ ID NO:11] or JC24 [SEQ ID NO: 12] 24-mer annealed with 12-mer oligonucleotides possessing sequences that are complementary to the last 8 nt of the 3′-terminus of the 24-mer and the 4 bp overhang. These 4 bp overhang sequences are chosen so as to be complementary to the overhangs that are generated by the restriction endonuclease digestion. In addition, the last 3′-terminal nucleotide of the 24-mer adapter (i.e., A or C) is selected such that a functional restriction endonuclease recognition site is not regenerated when the adapter anneals to the digested cDNA.

While the sequences of the two adapters are distinct, they possess common 5′ sequences that allow the formation of structures that suppress PCR-mediated amplification of the shorter fragments.

Following the ligation of the adapters, the restriction endonucleases are heat inactivated and the reaction mixture is PCR amplified.

The cDNA fragments prepared as above can be size-fractionated, e.g., electrophoretic fractionation on agarose or polyacrylamide gels, or other types of gels comprised of a similar material. The cDNA fragments may then be physically excised in defined size ranges (i.e., as identified by size makers) and recovered from the excised gel fragments. Additionally, if the quantities of isolated cDNA fragments are low, they can be amplified, e.g., by PCR amplification For example, if the cDNA fragments are generated by Long Internal SeqCalling™ Chemistry protocol, they are amplified with J23 [SEQ ID NO:6] and X22 [SEQ ID NO:15] primers (either before or after fractionation) prior to cloning, as these cDNAs cannot be efficiently cloned into E. coli. Similarly, if the cDNA fragments are generated by Long 5′ SeqCalling™ Chemistry protocol, they can be amplified by J23 [SEQ ID NO:6] and RS [SEQ ID NO: 1] oligonucleotides (either before or after fractionation) prior to cloning, as these products cannot be efficiently cloned into E. coli.

When PCR amplification is used to amplify fragments, conditions are preferentially chosen to minimize non-productive hybridization events. It has been observed that DNA re-hybridization during the PCR amplification process (designated the “Cot effect”; see e.g., Mathieu-Daude, et al., 1996. Nucl. Acids Res. 24: 2080-2084) can inhibit amplification. This effect is particularly evident during later PCR amplification cycles, when a substantial concentration of the amplified product has accumulated and the primer concentration has been depleted. As a result, amplification in the later PCR cycles typically follow non-linear dynamics.

By manipulating PCR amplification reaction conditions, it is possible to markedly enhance the “Cot effect”, by the insertion of a slow-annealing step in between the denaturation and re-naturation steps in each PCR amplification cycle. The slow-annealing temperature is chosen so as to be above that of the primer-template melting temperature (Tₘ), but at or above that of the template-template Tₘ, thus favoring template-template annealing over template-primer annealing. For example, a 85-75° C. decrease in temperature at a 10° C./minute gradient can be utilized

Partitioning Methods

One or more of the following techniques, or combinations these techniques, can be used to normalize the abundance of RNA (or their cDNA counterpart) species within a given cell or tissue sample.

(i) Partitioning by restriction endonuclease digestion

A cDNA library can be partitioned into many different sets of fragments by digestion with different restriction enzyme pairs. Fragmentation of the same cDNA library with different sets of restriction enzymes, in different reaction vessels, results in segregated multiple partitions, i.e., each specific fragment will occur in only one partition. The digested fragments can be analyzed further, e.g., by direct sequencing, cloning of the digested fragments or sequencing, or one or more of these techniques.

If desired, the cDNA is digested into fragments of a length that is convenient for sequencing. Preferably, multiple different partitions, e.g., 10-100, 20-750, or 50-250 partitions are obtained.

(ii) Partitioning by fragment size or other physical property

Partitioning can also be performed using other separation methods that separate DNA molecules according to their physical characteristics. The methods can include, e.g., separation based on physical and/or biochemical properties (i. e., molecular weight/size, terminal nucleotide sequences, exact migratory pattern, and the like). Separation methods can include, e.g., gel electrophoresis, including agarose or polyacrylamide gel electrophoresis, high pressure liquid chromatography (HPLC), preparative-scale capillary electrophoresis, and similar methodologies.

In one embodiment, unique cDNAs that represent unique (i.e., not previously sequenced) fragments are selected based on their presence in a characteristic restriction enzyme fragment. In this process, a cDNA population is digested with restriction endonucleases, fractionated, and fragments in a desired size range are recovered. The recovered fragments are then ligated to a vector and transformed into an appropriate host, e.g., E. coli. Rather that being directly sequenced following the selection process, the DNA fragments are isolated and separated, e.g., sized using one or more sizing matrixes that separate the molecules as a function of their physical or biochemical properties. The embodiment is thus referrred to as “clone sizing”. Those recombinant clones that have an insert with characteristics not present in a reference database are determined to contain a unique DNA fragment. Preferably, only unique fragments are subsequently sequenced.

For example, a DNA fragment that is sized in this way possesses two pieces of information that serve as a unique identifier: (i) the identity of the restriction endonuclease used to generate the fragment, and (ii) the size of the fragment. With these two pieces of information, fragments are picked for subsequent nucleotide sequencing by searching for a specific fragment within a 0.2 basepair window. If a fragment is present in the window, the E. coli clone containing the fragment is re-arrayed on a liquid handling robot such as a Tecan Genesis or Packard Multiprobe device, and sequenced. When multiple fragments are present within the 0.2 bp window, only one is selected to be sequenced. Thus, by use of this sizing filter, sequencing of identical fragments is significantly lowered.

By sizing individual fragments and comparing the observed size to previously determined sequences, i.e., using a “sizing filter”, only fragments of unique lengths need to be sequenced.

To pre-size large numbers of fragments, the fragments can be initially pooled as a function of their expected size, so as to ensure the any fragment occurs in a minimum of at least three individual pools.

Size fractionation may be accomplished in a number of ways. One commonly utilized method is electrophoretic fractionation on agarose or polyacrylamide gels, or other types of gels comprised of a similar material. The cDNA fragments may then be physically excised in defined size ranges (i.e., as identified by size makers) and recovered from the excised gel fragments. Additionally, if the quantities of isolated CDNA fragments are low, they can be PCR amplified at this stage. For example, if the cDNA fragments are generated by Long Internal SeqCalling™ Chemistry protocol, described above, they must be amplified with J23 and X22 primers (either before or after fractionation) prior to cloning, as these cDNAs cannot be efficiently cloned into E. coli. Similarly, if the cDNA fragments are generated by Long 5′ SeqCalling™ Chemistry protocol, described above, they must be amplified by J23 and RS oligonucleotides (either before or after fractionation) prior to cloning, as these products cannot be efficiently cloned into E. coli.

(iii) Partitioning based on hybridization

Screening can be performed using a variety of methods that rely on hybridization between a probe sequence or sequences and a cDNA library. Members of the library containing a homologous sequence are then removed from the library. For example, a cDNA library can be brought into contact with a prepared library of known sequence in such a way that any sequence contained within the substrate library that is complimentary to any element of the subtraction library is removed or suppressed. This method obviates re-characterizing, e.g., re-sequencing, already characterized members of the cDNA population.

(iv) Amplification-associated partitioning

Partitioning can also be performed in association with amplification. In particular, partitioning can be carried out during PCR amplification of adapter-ligated cDNA fragments described above. During PCR-mediated amplification of mixtures of cDNA fragments, short fragments tend to be preferentially amplified relative to large fragments. PCR conditions can be adjusted to favor the formation of larger fragments within the PCR reaction to allow efficient preferential amplification of longer fragments.

Normally, two different primers are used in PCR amplification to prime the enzymatic activity of the polymerase at each terminus of the target sequence. Conversely, if primers with identical 5′ sequences are used, there is a tendency for the fragments to form lariat or pan-handle structures, due to intra-strand hybridization, which interferes with the amplification process. Because the probability of the two ends of a polymer (i.e., cDNA fragment) finding one another is inversely proportional to a fractional power of the polymer length, short fragments tend to form these lariat structures more readily than do longer ones. Accordingly, this effect is exploited in the amplification of long cDNA fragments. See U.S. Pat. No. 5,565,340, whose disclosure is incorporated herein by reference, in its entirety.

Long fragment amplification can be enhanced using DNA fragments to which have been ligated long adapter sequences as described above. Amplification is dependent upon a number of factors that can alter the ratio of a linear adapter structure, which is permissive for amplification, and a lariat-loop structure, which suppresses amplifications. The equilibrium constant associated with the formation of the suppressive and the permissive structures, and, therefore, the efficiency of suppression of particular DNA fragments during PCR, is primarily a function of the following factors: (i) differences in melting temperature of suppressive and permissive structures; (ii) position of the primer sequence within the adapter; (iii) the length of the target DNA fragments; (iv) PCR primer concentration; and (v) primary structure.

Analysis of Partitioned cDNA Molecules

Partitioned cDNA molecules are next analyzed by comparing the sequences to a reference nucleic acid or nucleic acids. To facilitate analysis of partitioned cDNA molecules, they can, if not subcloned previously, be ligated into an appropriate vector and transformed into cells by any applicable method.

The reference nucleic acid or nucleic acids can be any fragment for which sufficient information is available to unambiguously identify the partitioned cDNA molecule. The reference nucleic acid or nucleic acids can therefore be part of, e.g., sequence databases, or databases of other characteristics that unambiguously identify a nucleic acid. Examples of such characteristics include e.g., a compilation of fragment sizes associated with specific restriction enzymes for a particular gene. In some embodiments, partitioned nucleic acids will be sequenced. The partitioned sequences can be sequenced by any method known to the art and the resulting sequence data is analyzed by computer-based systems.

Suitable databases include publicly available databases that comprehensively record all observed DNA sequences. Such databases include, e.g., GenBank from the National Center for Biotechnology Information (Bethesda, Md.), the EMBL Data Library at the European Bioinformatics Institute (Hinxton Hall, UK) and databases from the National Center for Genome Research (Santa Fe, N. Mex.). However, any database containing entries for the sequences likely to be present in such a sample to be analyzed is usable in the further steps of the computer methods. Methods of searching databases are described in detail in e.g., U.S. Pat. No. 5,871,697, whose disclosure is incorporated herein by reference, in its entirety.

Table 1 below summarizes the various primers and adapters disclosed herein.

TABLE 1

| SEQ | | |
|---|---|---|
| ID NO: | Name | Sequence (from 5′ to 3′) |
| 1 | RS | CTCTCCGATG CAGGTGGC |
| 2 | RXC | AGCACACTCC AGCCTCTCTC CGAGCACATG CGACACTGAG TACTAC |
| 3 | RXA | AGCACACTCC AGCCTCTCTC CGAGCACATG CGACACTGAG TACTAA |
| 4 | RJC | AGCACACTCC AGCCTCTCTC CGAACCGACG TCGAATATCC ATGCAGC |
| 5 | RJA | AGCACACTCC AGCCTCTCTC CGAACCGACG TCGAATATCC ATGCAGA |
| 6 | J23 | ACCGACGTCG AATATCCATG CAG |
| 7 | R23 | AGCACACTCC AGCCTCTCTC CGA |
| 8 | NR17 | AGCACACTCC AGCCTCT |
| 9 | RA24 | AGCACACTCC AGCCTCTCTC CGAA |
| 10 | RC24 | AGCACACTCC AGCCTCTCTC CGAC |
| 11 | JA24 | ACCGACGTCG AATATCCATG CAGA |
| 12 | JC24 | ACCGACGTCG AATATCCATG CAGC |
| 13 | dT-R | AGCACACTCC AGCCTCTCTC CGA |
| 14 | | AGCACACTCC AGCCTCTCTC CGATTTTTTT TTTTTTTTTT TTT |

### EXAMPLES

The invention will be further described in the following examples, which do not limit the scope of the invention described in the claims. Examples 1-6 collectively describe the synthesis and amplification of cDNA subfractions enriched for the 5′ terminal sequences of mRNA molecules. Example 7 describes clone sizing.

### Example 1

5′ cDNA Synthesis—Phosphatase/pyrophosphate Digestion

For each reaction, 2.5 μg mRNA (do not exceed 3 μg total) is added to H₂O so as to provide a total volume of 73.5 μl. This mixture is then heated to 65° C. for 10 minutes, and quick-cooled on ice. The CIAP Cocktail (see below) is made as follows:

### CIAP Cocktail

| | | | |
|---|---|---|---|
| For each reaction: | 10 μl 10x CIAP buffer | 110 | μl |
| | 2.5 μl RNasin (Promega) x 11 | 27.5 | μl |
| | 10 ∞l 0.1M DTT | 110 | μl |
| | 4 μl 0.01 U/μl CIAP* | 35 | μl |

1) 26.5 μl of the above enzyme mixture is added to each 3 μl mRNA to give a total volume of 30.5 μl. 73.5 μl of the RNA mix is then added to give a final volume of 100 μl.

2) Incubate at 37° C. for 40 minutes.

3) Add 100 μl TE buffer (10 mM Tris pH 8.0; 0.1 mM EDTA).

4) Add 200 μl Acid-Phenol.

5) Mix vigorously.

6) Add 200 μl Chloroform-Isoamyl Alcohol (24:1 v/v).

7) Mix vigorously.

8) Centrifuge in a microfuge at maximum speed for 10 minutes.

9) Remove supernatant and transfer to new tube. Discard bottom layer.

10) Repeat steps 4-9 (only for CIAP treatment, not in later steps).

11) Add 2 μl ssDNA carrier and 20 μl 3 M Sodium Acetate to each tube.

12) Vortex 10 seconds and add 440 μl of absolute ethanol.

13) Vortex 10 seconds and incubate at least 30 minutes at 80° C.

14) Centrifuge samples at 13,200×g for 15 minutes.

15) Wash nucleic acid pellets with 70% ethanol and air-dry pellet.

16) Dissolve nucleic acid pellet in 70 μl water and cool on ice.

17) Centrifuge for 10-15 seconds at maximum speed.

18) Transfer contents of tubes to 8-strip tubes.

19) Add 30 μl TAP cocktail (see below).

### TAP Cocktail

| | | | |
|---|---|---|---|
| For each reaction: | 10 μl 10x TAP buffer | 110 | μl |
| | 2.5 μl RNasin x 11 | 27.5 | μl |
| | 15.5 μl H₂O | 170.5 | μl |
| | 2.0 μl 10 U/μl TAP | 22 | μl |
| | (Epicenter) | | |

20) Add 30 μl of above mixture to each 70μl CIAP-treated sample for a total volume of 100 μl.

21) Incubate at 37° C. for 45 minutes.

22) Repeat Phenol/Chloroform extraction and precipitation as above in steps 6-9 and then 11-15 (do not resuspend pellet).

### Example 2

5′ CDNA Synthesis: DNA-RNA Hybrid Primer Ligation

1) Transfer samples from Example 1 to 8-strip tubes.

2) Resuspend pellet in Ligation Cocktail (see below).

### Ligation Cocktail

| | | | |
|---|---|---|---|
| For each reaction: | 3 μl 10 mM ATP | 33 | μl |
| | 1 μl RNasin x 11 | 11 | μl |
| | 4.5 μl H₂O | 49.5 | μl |
| | 2 μl R-BAP-TAP DNA/RNA | 22 | μl |
| | hybrid oligomer | | |

3) Add 10.5 μl of above mixture to each pellet, dissolve pellet completely at room temperature by (preferably) tapping the tube or vortexing if needed.

4) Make an enzyme mix as follows:

### Enzyme Mixture

| | | | |
|---|---|---|---|
| For each reaction: | 30 μl H₂O | 330 | μl |
| | 12 μl 5x DNA Ligase Buffer | 132 | μl |
| | (Life Tech) x 11 | | |
| | 1.5 μl RNasin | 16.5 | μl |
| | 6 μl T₄ RNA Ligase | 66 | μl |
| | (Life Tech.) | | |

### Total reaction volume 60 μl

5) Incubate overnight at 20° C.

6) Repeat Phenol/Chloroform and precipitation as above in CIP/TAP Cocktail protocol steps 6-9 and 11-15 (do not resuspend pellet).

### Example 3

5′ cDNA Synthesis: cDNA First-Strand Synthesis

1) Resuspend cDNA pellet in Random Hexamer Cocktail (see below).

### Random Hexamer Cocktail

| | | | |
|---|---|---|---|
| For each reaction: | 10 μl H₂O x 11 | 110 | μl |
| | 0.5 μl random hexamer | 5.5 | μl |
| | (dN₆-5′-Phosphate, | | |
| | 100 μM) | | |
| | 5 μl Oligo-(dT) | 55 | μl |
| | (dT₃₀VN-5′Phosphate, | | |
| | 100 μM) | | |

2) Add 15.5 μl of above mixture to each tube and resuspend pellet.

3) Heat at 70° C. for 10 minutes and quick-cool on ice.

4) Make First-Strand Synthesis Cocktail as follows (see below).

### First-Strand Synthesis Cocktail

| | | | |
|---|---|---|---|
| For each reaction: | 6 μl 5x First-Strand Buffer | 66 | μl |
| | 3 μl 10 mM dNTPs | 33 | μl |
| | 3 μl 100 mM DTT x 11 | 33 | μl |
| | 1 μl RNase Inhibitor | 11 | μl |

5) Add 13 μl of the above mixture to each 15.5 μl sample to give a total volume of 28.5 μl.

6) Incubate at 37° C. for 2 minutes.

7) Add 1.5 μl SuperScript II RT to each reaction for a total volume of 30 μl.

8) Incubate at 37° C. for 10 minutes.

9) Incubate at 42° C. for 1 hour.

10) Incubate at 16° C.

11) Add 40 μl of the following DNA Ligase Mixture (see below) to each reaction tube for a total volume of 70 μl.

### E. coli DNA Ligase Mixture

| | | | |
|---|---|---|---|
| For each reaction: | 4 μl 10x E. coli Ligase | 44 | μl |
| | Buffer x 11 | | |
| | 33 μl H₂O | 330 | μl |
| | 3 μl E. coli DNA Ligase | 33 | μl |
| | (10 U/μl) | | |

12) Continue incubation at 16° C. for 2 hours.

### Example 4

5′ CDNA Synthesis: Removal of Non-ligated Primers

While the above 2 hour incubation described in Example 3 is progressing, prepare one Boehringer-Mannheim Quick-Spin G-50 columns per reaction as follows:

1) Mix the resin bed well by inverting the columns repeatedly.

2) Remove the top cap first, and then the bottom cap. This avoids bubble formation and resultant poor performance of the spin-column.

3) Stand column vertically and allow to drain completely.

4) Add 0.75 ml of 10 mM Tris (pH 7.5) to the top of the bed without disturbing. If the bed becomes disturbed, pipette the solution up and down slowly to mix the bed uniformly and allow the bed to re-settle so as to form a uniform surface.

5) Stand column vertically and allow to drain completely.

6) Place the columns into a 15 ml conical centrifuge tube with the vendor's associated collector tube beneath the spin-column to collect the sample.

7) Centrifuge spin-column at 1000-1200×g for 2 minutes.

8) Remove spin-column with a forceps and remove the tube with flow through and discard.

9) Carefully load the sample to the top center of the spin-column.

10) Wash the sample tube with 20 μl H₂O and load on the same column.

11) Place a new collection tube beneath each spin-column and centrifuge at 1000-1200×g for 4 minutes.

12) Remove spin-columns and collect the flow-through into new, labeled tubes.

13) Total sample volume will be approximately 105 μl.

### Example 5

5′ CDNA Synthesis: RNase (H, A, and T₁) Treatment

1) To each reaction described in Example 4 add Second-Strand Reaction Buffer (see below).

### Second-Strand Reaction Buffer

| | | | |
|---|---|---|---|
| For each reaction: | 3 μl 100 mM DTT | 33 | μl |
| | 6 μl First-Strand Buffer | 33 | μl |
| | 30 μl Second-Strand | 330 | μl |
| | Buffer x 11 | | |
| | 6 μl H₂O | 66 | μl |

2) Add 45 μl of the above mixture to each 105 μl sample to give a total volume of 150 μl.

3) Add 2 μl of RNase H to each sample.

4) Incubate at 37° C. for 30 minutes to nick the RNA in RNA/DNA hybrids.

5) Make an RNase Mixture comprising: 22 μl RNase H, 44 μl RNase Cocktail (Ambion; available as an RNase A and RNase T₁ mixture).

6) Heat samples to 95° C. for 2 minutes.

7) Slow cool down to 37° C. and continue incubation.

8) Add 3 μl RNase Mixture to each of the cDNAs, mix by pipetting up and down.

9) Continue incubation at 37° C. for an additional 10 minutes.

10) Heat samples to 95° C. for 2 minutes.

11) Slow cool down to 37° C. and continue incubation.

12) Add an additional 3 μl of RNase Mixture to each of the cDNAs, mix by pipetting up and down.

13) Continue incubation at 37° C. for an additional 15 minutes.

14) Repeat Phenol/Chloroform extraction and precipitation as above in steps 6-9 and then 11-15.

15) Dissolve pellet in 20 μl H₂O.

16) Remove a 5 μl aliquot for Second-Strand (see below) synthesis for producing 5′-cDNA for SeqCalling™ Chemistry Protocol.

### Example 6

Second-Strand Synthesis for Producing 5′-cDNA for SeqCalling™ Chemistry

1) Generate PCR Mixture (see below) as follows:

### PCR Mixture

| | | | |
|---|---|---|---|
| For each reaction: | 5 μl 10x PCR Buffer x 11 | 55 | μl |
| | 1 μl 10 mM dNTPs | 5.5 | μl |
| | 1 μl 10 μM R17 Primer | 5.5 | μl |
| | 37.5 μl H₂O | 412.5 | μl |
| | 0.5 μl Advantage Polymerase | 5.5 | μl |

2) Add 45 μl of the above mixture to each 5 μl sample, for a total volume 50 μl.

3) Heat samples as per protocol below, making sure that the sample tubes are placed in the thermocycler only after it has reached>80° C.

| | | |
|---|---|---|
| 94° C. for 2 minutes | \| | |
| 55° C. for 2 minutes | \| | x 1 Cycle ONLY |
| 72° C. for 60 minutes | \| | (Cycle designated KM-AD-2N) |
| 4° C. for long-term storage | | |

4) Warm reaction tubes to 37° C.

5) Make SAP Cocktail (see below) as follows

### SAP Cocktail

| | |
|---|---|
| For each reaction: | |
| 12 μl 10x SAP Buffer × 11 | 132 μl |
| 5 μl H₂O | 55 μl |
| 3 μl Shrimp Alkaline Phosphatase (SAP; 1 U/μl) | 33 μl |

6) Add 20 μl of SAP Cocktail to each reaction.

7) Heat to 37° C. for 30 minutes.

8) Purify samples by Qiagen 96-well plate as manufacture's protocol.

9) Elute cDNAs in 100 μl 10 mM Tris-HCl buffer and proceed with fluorometry.

### Example 7

Clone Sizing

SeqCalling™ Chemistry products generated in any of Examples 1-6 are diluted and re-amplified. Fractionation is then performed by electrophoresising the re-amplified sample on an agarose gel using MetaPhor agarose (FMC). After the electrophoresis, the gel is physically cut into a total of 48 fractions. 24 of the fractions are derived from a 4% MetaPhor gel, and correspond to the lower molecular weight fractions; whereas the other 24 fractions derived from the 3% MetaPhor gel, correspond to the upper molecular weight fractions.

Following the elution of the DNA from the gel fractions, the DNA fragments are ligated into a vector with the TOPO-TA cloning vector (Invitrogen). These plasmids are then transformed into E. coli. The transformed bacterial cells are plated onto petri dishes and grown to a size that allows automated colony picking. A suitable number of colonies/fraction are selected so as to ensure a statistically accurate representation of the DNA fragments contained within the fraction (i.e., suitable numbers of picked colonies/fraction are 48 or 96). Following the incubation of the selected clones, the fragment contained within each individual clone are sized using the proprietary MegaBACE system, or an equivalent. Sizing is performed with multiple clones/lane. This multiplexing allows sizing to be performed in a cost and time efficient manner. The multiplexing is performed with a liquid handling robot (e.g., Matrix PlateMate). After running the multiplexed fragments on MegaBACE, and correlating the size of the fragment with the E. coli clone containing the insert, the fragments are analyzed to determine suitability for sequencing.

### Example 8

Comparison of Clone Complexity with and without Use of a Sizing Step

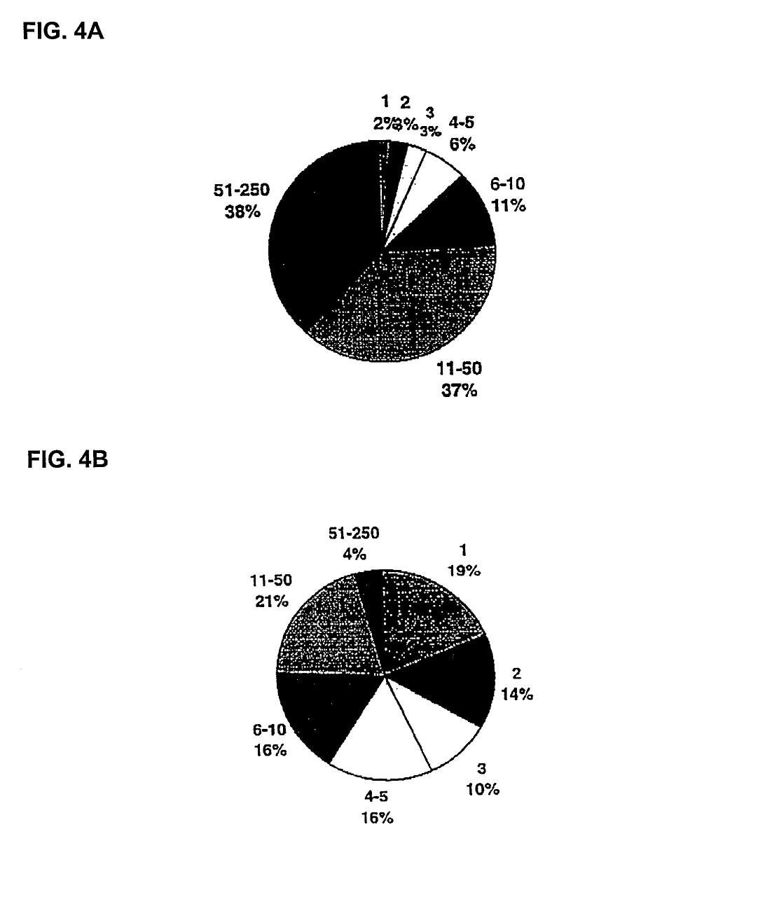

The effect of using a clone sizing step on the complexity, i.e., the representation of rarely transcripts, of the resulting clones, is shown in FIGS. 4A and 4B. In FIG. 4A, no sizing step was used, while clone sizing was used in the identification of the clones shown in FIG. 4B. Shown in the figures is a comparison of the frequencies (expressed in percentage) of clones derived from transcripts present at varying levels. The outer numbers represent the prevalence of a particular clone sequenced, and the inner numbers represents the percentages of the total number of clones sequenced that fall into this abundance class. As illustrated in FIG. 4A, the sequencing results that were obtained without the use of the sizing filter demonstrated that only a small percentage of the total number of fragments that were sequenced were included low copy number fragments (i. e., singletons, duplicates, and triplicates). Specifically, singletons were found to comprise only 2% of the total number of fragments sequenced, while fragments that were present at greater than 51 copies comprised 38% of the total fragments sequenced. In contrast, as illustrated in FIG. 4B, the sequencing results that were obtained with the use of the sizing filter were enriched for clones from low abundance transcripts (i.e., singletons, duplicates, and triplicates). These clones constituted approximately 33% of the total fragments sequenced. In contrast, without the use of this sizing filter, these fragments were found to only comprised a total of 8% of the sequencing results.

### Equivalents

Although particular embodiments have been disclosed herein in detail, this has been done by way of example for purposes of illustration only, and is not intended to be limiting with respect to the scope of the appended claims that follow. In particular, it is contemplated by the inventor that various substitutions, alterations, and modifications may be made to the invention without departing from the spirit and scope of the invention as defined by the claims. For example, the selection of the specific tissue(s) or cell line(s) that is to be utilized in the practice of the present invention is believed to be a matter of routine for a person of ordinary skill in the art with knowledge of the embodiments described herein.

What is claimed is:

1. A method of screening a population of nucleic acids for a novel sequence, the method comprising:
providing a population of nucleic acid sequences;
partitioning said population into one or more subpopulations of nucleic acids;
identifying a first nucleic acid sequence in the subpopulation of nucleic acid sequences; and
comparing the first nucleic acid sequence to a reference nucleic acid sequence or sequences, wherein the absence of the first nucleic acid sequence in the reference nucleic acid or nucleic acid sequences indicates the first nucleic acid is a novel nucleic acid sequence.

2. The method of claim 1, wherein said DNA population is a cDNA population derived from a population of RNA molecules.

3. The method of claim 2, further comprising partitioning the RNA molecules.

4. The method of claim 2, wherein said cDNA population is derived from the 5′ ends of the RNA molecules.

5. The method of claim 2, wherein said cDNA population is derived from the interior regions of the RNA molecules.

6. The method of claim 2, wherein said cDNA population is derived from the 3′ ends of the DNA molecules.

7. The method of claim 2, wherein said partitioning step comprises hybridization of a probe nucleic acid sequence to the population of nucleic acids.

8. The method of claim 2, wherein said partitioning step comprises digesting the cDNA molecules with one or more restriction enzymes.

9. The method of claim 8, further comprising ligating adapter oligonucleotides to the termini of the digested cDNA molecules.

10. The method of claim 9, further comprising amplifying the ligation products.

11. The method of claim 8, further comprising separating the amplified products.

12. The method of claim 11, wherein said separating is by gel electrophoresis.

13. The method of claim 11, wherein the first nucleic acid sequence is identified by comparing the size of one or more digestion products produced by a member of the subpopulation of nucleic acids to the sizes of fragments generated by the same restriction enzyme or enzymes in said reference nucleic acid or nucleic acids.

14. The method of claim 11, further comprising
recovering one or more size-separated digestion products;
reamplifying the recovered products; and
separating the reamplified products.

15. The method of claim 14, wherein said separating is by gel electrophoresis.

16. The method of claim 15, wherein the first nucleic acid sequence is identified by comparing the size of one or more digestion products produced by a member of the subpopulation of nucleic acids to the sizes of fragments generated by the same restriction enzyme or enzymes in said reference nucleic acid or nucleic acids.

17. The method of claim 9, further comprising:
inserting the ligated adapter oligonucleotide into a cloning vector to form a vector-insert;
transforming the vector-insert into a suitable host;
culturing transformed host under conditions allowing for replication of the vector-insert;
recovering the vector-insert from said host; and
digesting the vector-insert with one or more restriction enzymes, thereby releasing said insert; and
comparing the size of the insert to sizes of fragments generated by the same restriction enzyme or enzymes in said reference nucleic acid or nucleic acids.

18. The method of claim 1, wherein comparing is by determining at least a portion of the nucleotide sequence of the first nucleic acid sequence and comparing the nucleotide sequence to the nucleotide sequence of one or more reference nucleic acids.

19. The method of claim 1, wherein comparing is by hybridizing the first nucleic acid sequence to one or more of the reference nucleic acid sequences.

20. A method for equalizing the representation of nucleic acids in a population of nucleic acids, the method comprising:
providing a population of nucleic acid sequences, wherein said population comprises a first nucleic acid and a second nucleic acid having a nucleic acid sequence distinct from the first nucleic acid, and wherein said first nucleic acid is present at a higher level in said population than said second population;
partitioning said population into one or more subpopulations of nucleic acids; and
comparing the levels of said first nucleic acid sequence to the levels of said second nucleic acid sequence in the subpopulation of nucleic acid sequences, wherein a lower level of the first nucleic acid sequence relative to the second nucleic acid sequence indicates the representation of said first and second nucleic acid sequences are normalized.

21. A method for producing a population of nucleic acid molecules enriched for 5′ regions of mRNA molecules, the method comprising:
providing a population of RNA molecules, said population including RNA molecules having a 5′ terminal Gppp cap structure and a 5′ terminal phosphate group;
contacting said population of RNA molecules with a phosphatase under conditions that result in removal of the 5′ terminal phosphate group while leaving the 5′ terminal Gppp cap structure intact;
inactivating said phosphatase;
contacting the population of RNA molecules with a pyrophosphatase under conditions that result in the removal of the 5′ terminal Gppp and the formation of a 5′ phosphate group;
annealing an oligonucleotide in the presence of an RNA ligase to form a hybrid molecule; and
forming a cDNA from said oligonucleotide.

22. A method of identifying an RNA sequence in a sample comprising a plurality of RNA sequences, the method comprising:
synthesizing cDNA copies of a plurality of RNA species to form a cDNA sample;
determining the size of one or more of said cDNA molecules in said cDNA sample;
comparing the size of said sample with the size of a reference nucleic acid; and thereby identifying the cDNA sequence.

23. The method of claim 22, wherein said cDNA molecules are digested with one or more restriction enzymes prior to the determining step.

24. The method of claim 23, further comprising ligating adapter oligonucleotides to the termini of the digested cDNA molecules prior to the determining step.

25. The method of claim 22, wherein said identifying step comprises comparing the size of one or more digestion products produced by one or more said cDNA molecules to a reference nucleic acid or nucleic acids.

26. A method of identifying an RNA sequence in a population of RNA sequences, the method comprising:
(a) removing 5′ terminal pppG from RNAs in said population to form a population of RNAs having terminal 5′ phosphate groups;
(b) ligating a linker oligonucleotide to the terminal 5′ phosphate groups of RNA molecules in said population of RNAs;
(c) synthesizing complementary cDNA molecules from said population of RNA molecules to form a cDNA sample;
(d) digesting said complementary cDNA molecules with at least one restriction enzyme;
(e) ligating an adapter molecule to the digested cDNA molecules;
(f) amplifying the molecules produced in step (e);
(g) identifying the amplified molecules of step (f); and
(h) comparing the amplified molecules to one or more reference nucleic acids.
